(19) United States
(12) Patent Application Publication
(10) Pub. No.: US 2025/0090058 A1
(43) Pub. Date: Mar. 20, 2025

(54) OPTO-ELECTRONIC HARDWARE CONFIGURATIONS FOR IMPLANTABLE CHEMICAL SENSORS

(71) Applicant: Cardiac Pacemakers, Inc., St. Paul, MN (US)

(72) Inventors: Michael J. Kane, St. Paul, MN (US); Yingbo Li, Woodbury, MN (US); James Michael English, Cahir (IE)

(21) Appl. No.: 18/882,966

(22) Filed: Sep. 12, 2024

Related U.S. Application Data
(60) Provisional application No. 63/538,366, filed on Sep. 14, 2023.

(51) Int. Cl. A61B 5/1459; A61B 5/00

(52) U.S. Cl. CPC A61B 5/1459; A61B 5/6861; A61B 2562/0238

(57) ABSTRACT

Embodiments herein relate to implantable chemical sensors. In an embodiment, an implantable optical chemical sensor includes a circuit board, at least one optical emitter, and at least one optical detector. The optical emitter can be disposed on the circuit board along with the optical detector. The optical chemical sensor can also include a floor plate and a well plate, the well plate defining at least one well and the well plate disposed over the floor plate. The well plate can include a vertical facet and a beveled facet disposed on a perimeter edge of the well plate. A feed-through flange can define a window with the vertical facet of the well plate fitting into the window. The optical emitter can emit light through the floor plate and into the well plate which is then redirected by the beveled facet into the well. Other embodiments are also included herein.

This application claims the benefit of U.S. Provisional Application No. 63/538,366, filed Sep. 14, 2023, the content of which is herein incorporated by reference in its entirety.

### FIELD

Embodiments herein relate to implantable chemical sensors.

### BACKGROUND

Data regarding physiological analytes are highly relevant for the diagnosis and treatment of many conditions and disease states. As one example, potassium ion concentrations can affect a patient's cardiac rhythm. Therefore, medical professionals frequently evaluate physiological potassium ion concentration when diagnosing a cardiac rhythm problem. However, measuring physiological concentrations of analytes, such as potassium, generally requires drawing blood from the patient. Blood draws are commonly done at a medical clinic or hospital and therefore generally require the patient to physically visit a medical facility. As a result, despite their significance, physiological analyte concentrations may only be measured sporadically.

### SUMMARY

Embodiments herein relate to implantable chemical sensors. In a first aspect, an implantable optical chemical sensor can be included having a circuit board, at least one optical emitter, and at least one optical detector. The at least one optical emitter can be disposed on the circuit board along with the least one optical detector. The optical chemical sensor can also include a floor plate and a well plate, the well plate defining at least one well. The well plate can include a vertical facet and a beveled facet, wherein the beveled facet and the vertical facet can be disposed on at least one perimeter edge of the well plate. The well plate can be disposed over the floor plate. A feed-through flange can be included defining a window. The vertical facet of the well plate can fit into the window. The at least one optical emitter can be configured to emit light through the floor plate and into the well plate that can be then redirected by the beveled facet of the well plate into the at least one well.

In a second aspect, in addition to one or more of the preceding or following aspects, or in the alternative to some aspects, the at least one well can have a polished inner surface.

In a third aspect, in addition to one or more of the preceding or following aspects, or in the alternative to some aspects, the beveled facet can be disposed at an angle of 20 to 70 degrees with respect to a surface of the vertical facet.

In a fourth aspect, in addition to one or more of the preceding or following aspects, or in the alternative to some aspects, the well plate can define at least two wells.

In a fifth aspect, in addition to one or more of the preceding or following aspects, or in the alternative to some aspects, the implantable chemical sensor can further include a sensor element, wherein the sensor element can be configured to fit into the at least one well.

In a sixth aspect, in addition to one or more of the preceding or following aspects, or in the alternative to some aspects, the implantable chemical sensor can further include a coating, wherein the coating can be disposed over the beveled facet and configured to internal reflection of light off the beveled facet.

In a seventh aspect, in addition to one or more of the preceding or following aspects, or in the alternative to some aspects, the floor plate includes a low-index glass.

In an eighth aspect, in addition to one or more of the preceding or following aspects, or in the alternative to some aspects, the least one optical detector can be a photo-diode.

In a ninth aspect, in addition to one or more of the preceding or following aspects, or in the alternative to some aspects, the implantable optical chemical sensor can further include a top housing shell, the top housing shell can define a sensor window. The feed-through flange can fit into the sensor window.

In a tenth aspect, in addition to one or more of the preceding or following aspects, or in the alternative to some aspects, a top surface of the feed-through flange fits flush with a top surface of the top housing shell.

In an eleventh aspect, in addition to one or more of the preceding or following aspects, or in the alternative to some aspects, the implantable optical chemical sensor can further include a bottom housing shell, wherein the bottom housing shell interfaces with the top housing shell along lateral edges thereof.

In a twelfth aspect, an implantable optical chemical sensor can be included having a circuit board, at least one optical emitter, at least one optical detector, a floor plate and a well plate. The at least one optical emitter can be disposed on the circuit board. The well plate can define at least one well. The well plate can be disposed over the floor plate. The optical chemical sensor can include at least one prism, wherein the at least one prism can be arranged to receive light from the at least one optical emitter and direct the same into the well plate. The optical chemical sensor can include a least one optical detector, wherein the least one optical detector can be disposed on the circuit board. The optical chemical sensor can also include a feed-through flange, the feed-through flange defining a window.

In a thirteenth aspect, in addition to one or more of the preceding or following aspects, or in the alternative to some aspects, the at least one well can have a polished inner surface.

In a fourteenth aspect, in addition to one or more of the preceding or following aspects, or in the alternative to some aspects, the well plate can define at least two wells.

In a fifteenth aspect, in addition to one or more of the preceding or following aspects, or in the alternative to some aspects, the optical chemical sensor can further include a prism holder, wherein the prism holder can be configured to hold a prism and fit over at least one optical emitter thereby aligning the same.

In a sixteenth aspect, in addition to one or more of the preceding or following aspects, or in the alternative to some aspects, the optical chemical sensor can further include a sensor element, wherein the sensor element can be configured to fit into the at least one well.

In a seventeenth aspect, in addition to one or more of the preceding or following aspects, or in the alternative to some aspects, the floor plate includes a low-index glass.

In an eighteenth aspect, in addition to one or more of the preceding or following aspects, or in the alternative to some aspects, the least one optical detector can be a photo-diode.

In a nineteenth aspect, in addition to one or more of the preceding or following aspects, or in the alternative to some aspects, the implantable optical chemical sensor can further include a top housing shell, the top housing shell defining a sensor window, and wherein the feed-through flange fits into the sensor window.

In a twentieth aspect, in addition to one or more of the preceding or following aspects, or in the alternative to some aspects, a top surface of the feed-through flange fits flush with a top surface of the top housing shell.

In a twenty-first aspect, in addition to one or more of the preceding or following aspects, or in the alternative to some aspects, the optical chemical sensor can further include a bottom housing shell, wherein the bottom housing shell interfaces with the top housing shell along lateral edges thereof.

In a twenty-second aspect, an implantable optical chemical sensor can be included having a circuit board, at least one optical emitter, at least one optical detector, and a well plate. The at least one optical emitter and at least one optical detector can be disposed on the circuit board. The implantable optical chemical sensor can include at least one prism. The well plate can define at least one well. The well plate can also define at least one prism alignment notch, wherein the at least one prism alignment notch can be configured to receive at least a portion of the at least one prism or a structure connected thereto thereby aligning the prism with the well plate. The implantable optical chemical sensor can also include a feed-through flange, the feed-through flange defining a window. The at least one prism can be arranged to receive light from the at least one optical emitter and direct the same into the well plate.

In a twenty-third aspect, in addition to one or more of the preceding or following aspects, or in the alternative to some aspects, the at least one well can have a polished inner surface.

In a twenty-fourth aspect, in addition to one or more of the preceding or following aspects, or in the alternative to some aspects, the well plate can define at least two wells.

In a twenty-fifth aspect, in addition to one or more of the preceding or following aspects, or in the alternative to some aspects, the implantable optical chemical sensor can further include a prism holder, wherein the prism holder can be configured to hold a prism and fit over at least one optical emitter thereby aligning the same.

In a twenty-sixth aspect, in addition to one or more of the preceding or following aspects, or in the alternative to some aspects, the implantable optical chemical sensor can further include a sensor element, wherein the sensor element can be configured to fit into the at least one well.

In a twenty-seventh aspect, in addition to one or more of the preceding or following aspects, or in the alternative to some aspects, the implantable optical chemical sensor can further include a floor plate, wherein the well plate can be disposed over the floor plate.

In a twenty-eighth aspect, in addition to one or more of the preceding or following aspects, or in the alternative to some aspects, the floor plate includes a low-index glass.

In a twenty-ninth aspect, in addition to one or more of the preceding or following aspects, or in the alternative to some aspects, the least one optical detector can be a photo-diode.

In a thirtieth aspect, in addition to one or more of the preceding or following aspects, or in the alternative to some aspects, the implantable optical chemical sensor can further include a top housing shell, the top housing shell defining a sensor window. The feed-through flange can fit into the sensor window.

In a thirty-first aspect, in addition to one or more of the preceding or following aspects, or in the alternative to some aspects, a top surface of the feed-through flange fits flush with a top surface of the top housing shell.

In a thirty-second aspect, in addition to one or more of the preceding or following aspects, or in the alternative to some aspects, the implantable optical chemical sensor can further include a bottom housing shell, wherein the bottom housing shell interfaces with the top housing shell along lateral edges thereof.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an implantable medical device with an optical chemical sensor in accordance with various embodiments herein.

FIG. 2 is an exploded view of portions of an implantable medical device in accordance with various embodiments herein.

FIG. 3 is a schematic elevation view of portions of an implantable medical device in accordance with various embodiments herein.

FIG. 4 is a schematic elevation view of portions of an implantable medical device in accordance with various embodiments herein.

FIG. 5 is a schematic view of a floor plate and a well plate of an optical chemical sensor in accordance with various embodiments herein.

FIG. 6 is a schematic cross-sectional view of a floor plate and a well plate of an optical chemical sensor in accordance with various embodiments herein.

FIG. 7 is a schematic plan view of an implantable optical chemical sensor in accordance with various embodiments herein.

FIG. 8 is a schematic side view of an implantable optical chemical sensor in accordance with various embodiments herein.

FIG. 9 is a schematic cross-sectional view of components of an optical chemical sensor in accordance with various embodiments herein.

FIG. 10 is a schematic cross-sectional view of components of an optical chemical sensor in accordance with various embodiments herein.

FIG. 11 is a schematic cross-sectional view of components of an optical chemical sensor in accordance with various embodiments herein.

FIG. 12 is a schematic plan view of an implantable optical chemical sensor in accordance with various embodiments herein.

FIG. 13 is a schematic perspective view of portions of an implantable optical chemical sensor in accordance with various embodiments herein.

FIG. 14 is a schematic elevation view of portions of an implantable optical chemical sensor element in accordance with various embodiments herein.

FIG. 15 is a perspective view of portions of an implantable chemical sensor element in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Data regarding physiological analytes are highly relevant for the diagnosis, treatment, and/or monitoring of many conditions and disease states. However, in vitro assays to determine physiological analyte values have substantial limitations. In contrast, implantable chemical sensors can be used to gather data about physiological analytes while a patient is away from a medical care facility and without needing to draw blood or another fluid from the patient.

However, implantable chemical sensors present various challenges. For example, the amount of space inside implanted devices is extremely limited. As such, one design challenge for implantable chemical sensors is to keep them as compact as possible. In addition, there is a need to be able to manufacture implantable chemical sensors at scale while ensuring that components are properly aligned with one another.

Embodiments herein relate to implantable medical devices and chemical sensors with a highly compact, space-efficient design and features promoting accurate alignment of components during assembly to allow efficient manufacturing of highly accurate chemical sensor devices. Embodiments herein can utilize planar construction designs including circuit boards and surface mount components thereon that can be easily and accurately mated with various optical components as described herein. As one example, an implantable optical chemical sensor herein can include a circuit board, at least two optical emitters disposed on the circuit board, a least one optical detector disposed on the circuit board, a floor plate, and a well plate defining at least one sensor well. The floor plate can be disposed under the well plate and can form the bottom of the sensor well. The well plate can also include a vertical facet and a beveled facet disposed on at least one perimeter edge of the well plate. A feed-through flange can define a window and the vertical facet of the well plate can fit into the window. The at least two optical emitters can be configured to emit light into the well plate (directly or via the floor plate) that can then be redirected by the beveled facet of the well plate into the at least one sensor well.

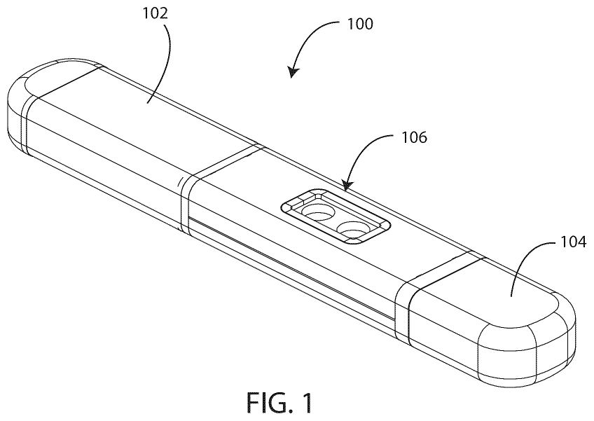

Referring now to FIG. 1, a schematic view of an implantable medical device 100 with an optical chemical sensor 106 is shown in accordance with various embodiments herein. In this example, the implantable medical device 100 includes a battery module 102. The battery module 102 can include an electrochemical cell disposed therein to provide power for the implantable medical device 100. In various embodiments herein, the electrochemical cell can be a primary lithium-manganese dioxide (Li anode/MnO₂ cathode) battery. However, other primary lithium battery chemistries are also contemplated herein. Other primary battery chemistries can include, but are not limited to, CFx, SVO, hybrid CFx/Mn02, hybrid CFx/SVO, and the like. In some embodiments, however, a rechargeable battery can be used. Recharging techniques herein can include RF charging, inductive charging, and the like. The implantable medical device 100 also includes a sensor module 104. The sensor module 104 can include an optical chemical sensor 106 as well as, optionally, other sensors and electronics.

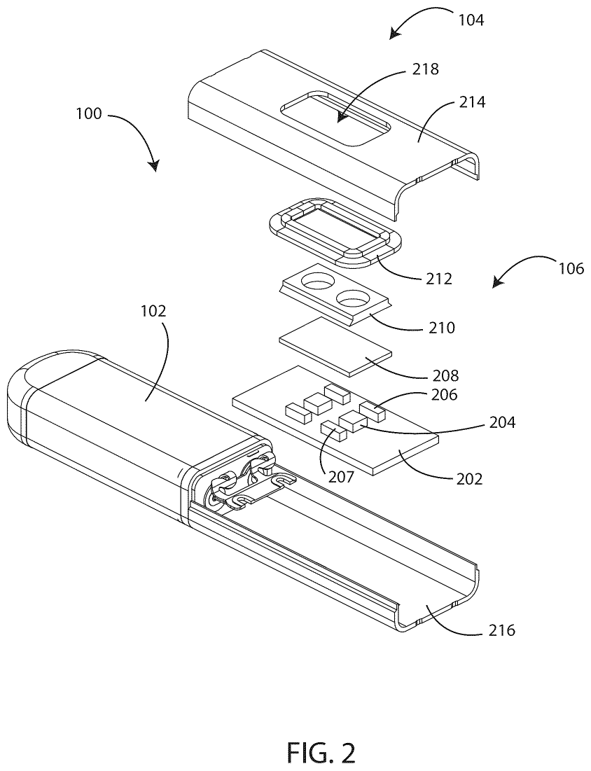

Referring now to FIG. 2, an exploded view is shown of portions of an implantable medical device 100 in accordance with various embodiments herein. As before, the implantable medical device 100 includes a battery module 102 and a sensor module 104. The sensor module 104 includes a top housing shell 214 and a bottom housing shell 216. The top housing shell 214 and a bottom housing shell 216 can be formed of a biocompatible electrically conductive material such as titanium or a titanium alloy. The bottom housing shell 216 can interface with the top housing shell 214 along lateral edges thereof. In various embodiments, the top housing shell 214 and the bottom housing shell 216 can be attached together (such as by welding or using another technique) along intersecting edges thereof, such as the lateral edges thereof. The top housing shell 214 and the bottom housing shell 216 can define a space there between to hold various components, including chemical sensor components herein.

Chemical sensor components of the implantable medical device 100 can include a circuit board 202, one or more optical detectors 204, one or more first optical emitters 206, and one or more second optical emitters 207.

The implantable medical device 100 also includes a floor plate 208, a well plate 210, and a feed-through flange 212. In various embodiments, the floor plate 208 and/or the well plate 210 itself can be formed of a glass, crystal, ceramic, polymer, or the like. In various embodiments, the floor plate 208 and/or the well plate 210 itself can be formed of a low-index glass, crystal, ceramic, or polymer, such as one having an index of refraction of 1.5 or less. In some embodiments, the refractive index of the floor plate 208 can be different than the refractive index of the well plate 210 in order to keep light within the well plate 210 after it has entered. The floor plate 208 and/or the well plate 210 can be formed using various techniques including machining, molding, or the like.

Components such as the optical detector(s) 204, first optical emitter(s) 206, and second optical emitter(s) 207 can be disposed on the circuit board 202 as surface mount hardware components promoting a compact configuration. Various options for the optical emitters and detectors are described below, but in some embodiments the first optical detector 204 can be a photo-diode and the optical emitter(s) 206, 207 can be light emitting diodes.

In various embodiments, the optical emitter(s) 206, 207 can be configured to emit light through the floor plate 208 and into the well plate 210. In embodiments where the floor plate 208 is omitted or does not extend over the optical emitters then the optical emitters can emit light directly into the well plate without passing through the floor plate 208. In some embodiments, the floor plate 208 and the well plate 210 can be physically integrated, but in other embodiments they are discrete components.

The well plate 210 can include a vertical facet (described further below) and a beveled facet (described further below). Light can be redirected by the beveled facet of the well plate 210 into at least one sensor element well defined by the well plate 210. A sensor element (not shown in FIG. 2, but described further below) can be configured to fit into the at least one sensor element well.

The feed-through flange 212 can fit into a sensor window 218 defined by the top housing shell 214. In various embodiments, a top surface of a feed-through flange 212 fits flush with a top surface of a top housing shell 214. In various embodiments, the feed-through flange 212 can be formed of a metal, a ceramic, a polymer, or the like. In some embodiments, the feed-through flange 212 can specifically be formed of a metal (such as titanium or a titanium alloy) to allow for the feed-through flange 212 to be welded into place within the sensor window 218 defined by the top housing shell 214. In various embodiments, the feed-through flange 212 can have rounded corners in order to facilitate attachment with the sensor window 218 while minimizing areas of concentrated stress.

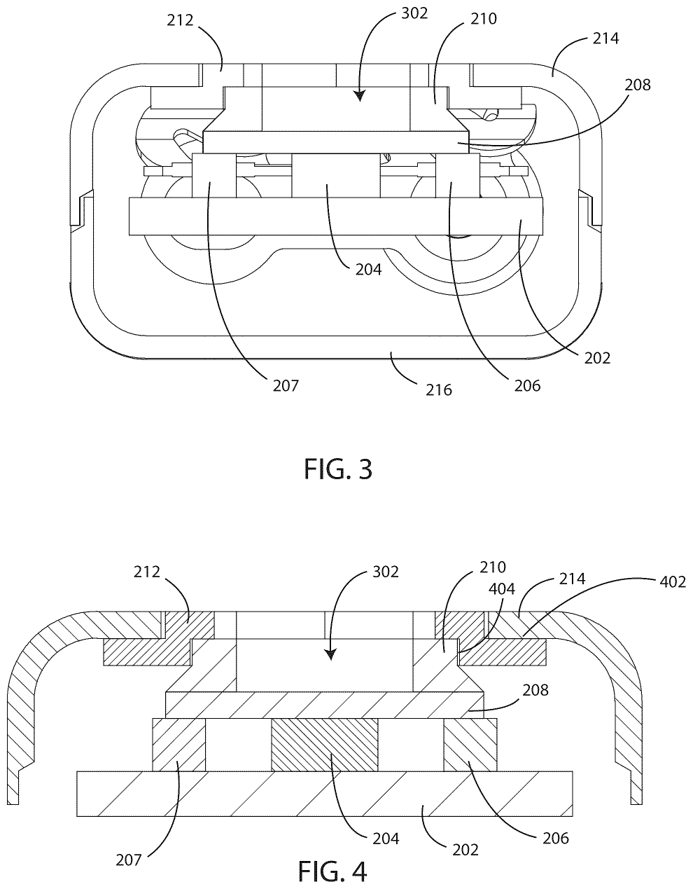

Referring now to FIG. 3, a schematic cross-sectional view of portions of an implantable medical device 100 are shown in accordance with various embodiments herein. As before, the implantable medical device 100 includes a top housing shell 214 and a bottom housing shell 216. The implantable medical device 100 also includes a circuit board 202. A first optical detector 204 and optical emitter(s) 206, 207 are disposed on the circuit board 202. The implantable optical chemical sensor also includes a floor plate 208 and a well plate 210. The well plate 210 includes a first sensor well 302. The implantable optical chemical sensor also includes a feed-through flange 212. The feed-through flange 212 fits into the top housing shell 214.

Referring now to FIG. 4, a schematic elevation view of portions of an implantable medical device 100 are shown in accordance with various embodiments herein. As before, the implantable medical device 100 includes a top housing shell 214, a circuit board 202, an optical detector 204, and optical emitter(s) 206, 207 disposed on the circuit board 202. The implantable optical chemical sensor also includes a floor plate 208, a well plate 210, and a feed-through flange 212 that fits into a sensor window of the top housing shell 214. In various embodiments, wherein the top surface of the feed-through flange 212 fits flush with a top surface of the top housing shell 214. In some embodiments, the feed-through flange 212 can include a recessed rim 402, onto which edges of the top housing shell 214 surrounding the sensor window 218 can rest. The feed-through flange 212 can also define an inner cavity 404 into which a portion of the well plate 210 (such as a vertical facet) can fit.

While not shown in FIG. 4, in some embodiments a protective cover layer that is also permeable to analytes of interest can be disposed over the outside of the chemical sensor, such as over the feed-through flange 212, over the sensor element within the sensor well, and/or over the exterior of the top housing shell 214. In some embodiments, the protective cover layer can be formed of a polymer such as polyHEMA (a polymer of 2-hydroxyethyl methacrylate) or another polymer.

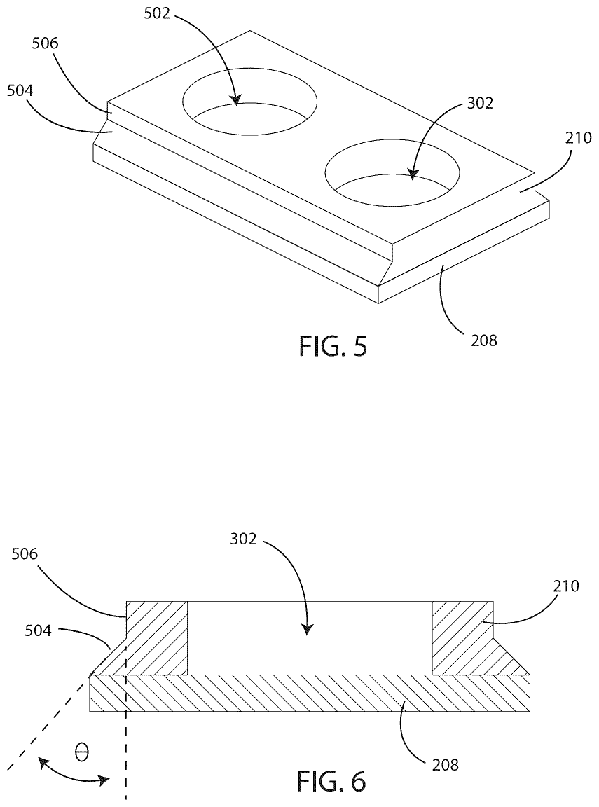

Referring now to FIG. 5, a schematic view of a floor plate 208 and a well plate 210 of an optical chemical sensor is shown in accordance with various embodiments herein. The well plate 210 includes a first sensor well 302. In this embodiment, the well plate 210 also includes a second sensor well 502. However, it will be appreciated that the well plate 210 can have any specific number of sensor wells (1, 2, 3, 4, 5, 6, 8, 10, etc.). As described further below, sensor elements can be placed in the wells and components of the sensor elements can interface with analytes of interest to create a response that can be detected through optical means described herein. In some embodiments, a particular well and the sensor element therein can be specific for a particular analyte of interest. In some embodiments, a particular well and the sensor element therein can be specific for more than one analyte of interest (such as in the case of a multi-modal sensor element). Analytes detected herein can include, but are not limited to, potassium, sodium, calcium, blood urea nitrogen (BUN), creatinine, and the like. In some embodiments, a pH sensor can also be included with the implantable device.

In various embodiments, the sensor wells can have a polished inner surface, such as to facilitate efficient passage of light from within the well plate 210 and into the sensor well. In some embodiments, a circular edge of the sensor well can have a lensing effect in order to focus light onto the sensor element disposed within the sensor well. In many embodiments, the sensor wells can be circular in shape, although other shapes are also contemplated herein such as oval shapes, square, rectangular, polygonal shapes, polygons with rounded corners, etc. In some embodiments, the sensor well can be semicircular with flattened sides (straight portions) on those sides adjacent to optical emitters, such as two opposed flattened sides. The sensor wells can be of various sizes, but can be big enough to accommodate a sensing element therein. In some embodiments, the sensor wells can be from 0.2 to 3 millimeters in diameter. In some cases, the sensor wells can be about 1.5 or 2 millimeters in diameter.

The well plate 210 includes a beveled facet 504 and a vertical facet 506. In various embodiments, the beveled facet 504 and a vertical facet 506 are disposed on at least one perimeter edge of a well plate 210. In some embodiments, the beveled facet 504 and a vertical facet 506 are disposed on two opposed perimeter edges of the well plate 210. In some embodiments, the beveled facet 504 is not on two perimeter edges of the well plate 210.

Referring now to FIG. 6, a schematic cross-sectional view of a floor plate 208 and a well plate 210 of an optical chemical sensor is shown in accordance with various embodiments herein. As before, the well plate 210 includes a first sensor well 302 along with a beveled facet 504 and a vertical facet 506. The beveled facet 504 can be angled so as to guide light coming up from the bottom of the well plate 210 so that it passes through the well plate 210 and towards the sensor wells 302, 502. In various embodiments, the beveled facet 504 can be disposed at an angle θ of 20 to 70 degrees with respect to a surface of the vertical facet 506. In various embodiments, the beveled facet 504 can be disposed at an angle θ of 40 to 50 degrees with respect to a surface of the vertical facet 506. However, in some embodiments, the vertical facet 506 can be omitted. In some embodiments the beveled facet 504 can be omitted.

In various embodiments, a coating (not shown in this view) can be disposed over the beveled facet 504 and configured to promote internal reflection of light off the beveled facet 504. For example, the coating can be reflective coating. The coating can be a metallized or silvered coating (such as one that is sputtered on). In some embodiments, an adhesive, such as an optically transparent adhesive, can be used to secure the well plate 210 to the floor plate 208.

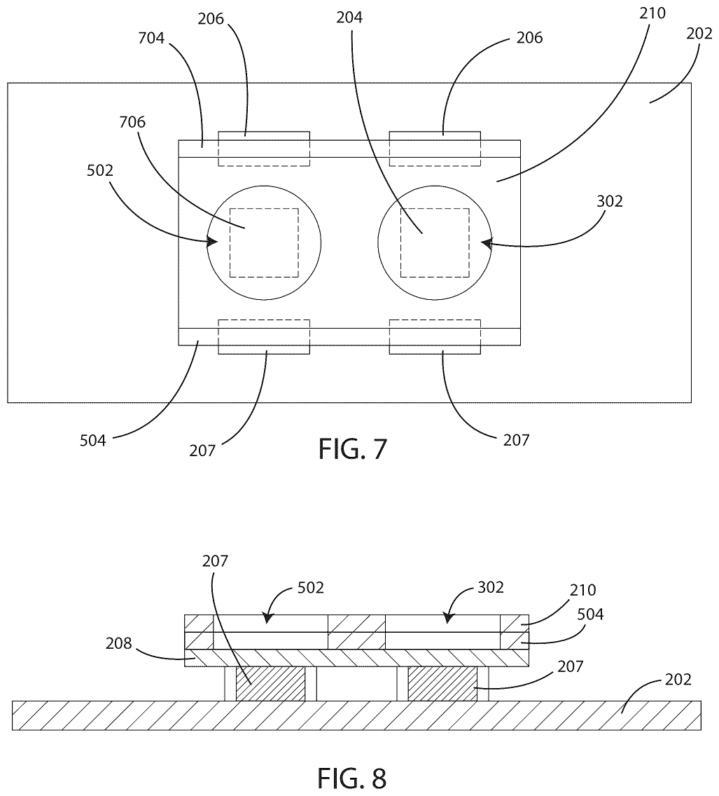

Referring now to FIG. 7, a schematic plan view of components of an implantable optical chemical sensor is shown in accordance with various embodiments herein. As before, the chemical sensor includes a circuit board 202, a first optical detector 204, first optical emitters 206, and second optical emitters 207. The implantable optical chemical sensor also includes a second optical detector 706. The implantable optical chemical sensor also includes a well plate 210. The well plate 210 defines a first sensor well 302 and a second sensor well 502. The well plate 210 also includes a first beveled facet 504 as well as a second beveled facet 704 disposed on an opposing side of the well plate 210 as the first beveled facet 504. Referring now to FIG. 8, a schematic side view of components of an implantable optical chemical sensor are shown in accordance with various embodiments herein. The implantable optical chemical sensor includes a circuit board 202, second optical emitters 207, floor plate 208, and well plate 210. The well plate 210 includes a first sensor well 302 and a second sensor well 502. The well plate 210 also includes first beveled facet 504. In some cases, the edges of the well plate 210 may not meet the edges of the floor plate 208.

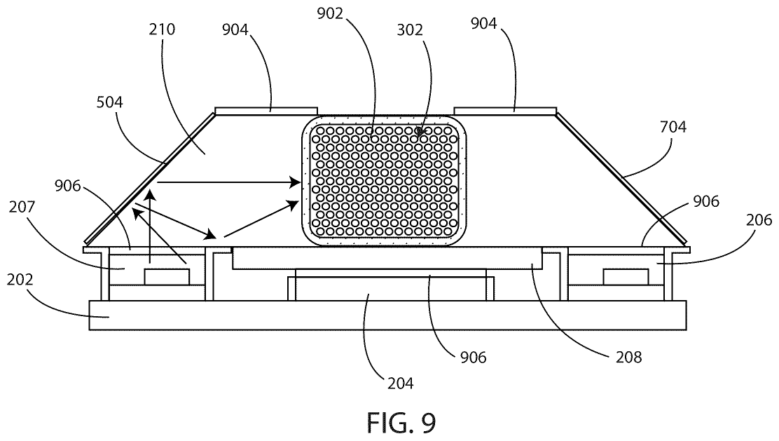

Devices herein can be configured to operate in scattering and/or transmission modes, with double ended or single ended light source configurations. Referring now to FIG. 9, a schematic cross-sectional view of components of an optical chemical sensor are shown in accordance with various embodiments herein. FIG. 9 serves as an example of an optical sensor working in a scattering mode. The implantable optical chemical sensor includes a circuit board 202, first optical detector 204, first optical emitter 206, and second optical emitter 207. The implantable optical chemical sensor also includes a floor plate 208 and a well plate 210. The well plate 210 includes a first beveled facet 504 and a second beveled facet 704.

In this example, the implantable optical chemical sensor also includes a mask coating 904. The mask coating 904 can be disposed on surfaces of the well plate 210, the floor plate 208, and/or other components herein. The mask coating 904 can be an opaque material and prevent ambient light from entering such components and can be formed of various materials. The mask coating 904 can be used to eliminate or reduce undesirable light leak paths. In some embodiments, the mask coating 904 can include carbon black and/or various pigments or components to render the coating opaque. In some embodiments, the mask coating 904 can be disposed on a top surface of the well plate 210, such as adjacent to the sensor well 302. In some embodiments, a mask coating can be disposed on a beveled facet 504. In some embodiments, a reflective material later can be disposed on a beveled facet 504, such as a metallized or silvered reflective coating (such as one that is sputtered on).

The implantable optical chemical sensor can also include an optically transparent adhesive 906. The optically transparent adhesive 906 can be used between various components of the implantable optical chemical sensor and can be used to adhere components together while not substantially attenuating light passing therethrough. In some embodiments, the optically transparent adhesive 906 can be one with an index of refraction approximately matching the components being joined, such as the well plate 210 and/or the floor plate 208. Optically transparent adhesives herein can include various acrylics, silicones, and the like.

The implantable optical chemical sensor also includes a sensor element 902. In various embodiments, the sensor element 902 can be configured to fit into at least one well. In some embodiments, the sensor element 902 can include an outer permeable layer (such as a polyHEMA layer) and sensor chemistry disposed therein, such as on beads as a carrier and/or dispersed in a polymeric matrix. Details of exemplary sensor elements are provided in greater detail below.

In operation, light emitted from the first optical emitter 206 and the second optical emitter 207 is directed toward the sensor element 902 within the sensor well 302. For example, light can be emitted up into the well plate 210, directly or in some embodiments after passing through floor plate 208 (in some embodiments, such as that shown in FIGS. 3-4, the floor plate 208 is disposed between the emitters and the well plate 210, while in other embodiments, such as that shown in FIG. 9, the floor plate 208 is not between the emitters and the well plate 210). Light originating from optical emitters 206, 207 can be guided toward the sensor well 302 by the beveled facets 504, 704. Such light can interface with the sensor element 902 and scatter including downward through the floor plate 208 and onto optical detector 204 for detection by the optical chemical sensor.

It will be appreciated that in some embodiments the positions of emitters and detectors as shown herein can be reversed. For example, an emitter (such as a polychromatic light source) can be located where optical detector 204 is shown in FIG. 9 and detectors (such as detectors with different wavelength sensitivities-using filters or the like) can be located where the optical emitters 206, 207 are shown in FIG. 9. In some embodiments, different wavelengths of light can be emitted sequentially by emitter(s) herein and magnitudes of light detected with the optical detector can be compared relative to one another.

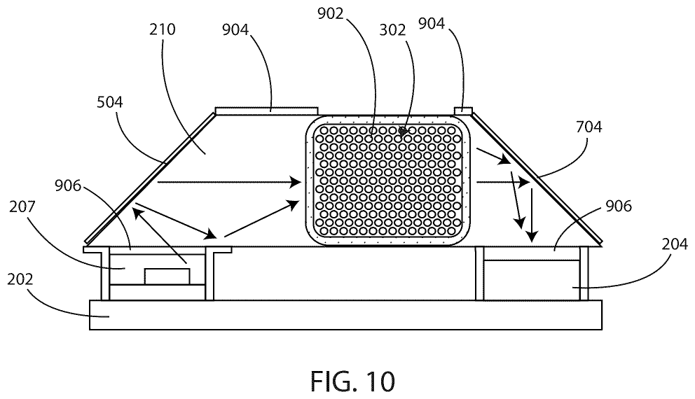

In some embodiments, the chemical sensor can operate in a transmission mode. In such an embodiment, light can interface with the sensor element and pass therethrough before being received by an optical detector. For example, referring now to FIG. 10, a schematic cross-sectional view of components of an optical chemical sensor is shown in accordance with various embodiments herein illustrating operation in a transmission mode. As before, the implantable optical chemical sensor includes a circuit board 202, an optical detector 204, an optical emitter 207, a well plate 210 with a first beveled facet 504 and a second beveled facet 704. The implantable optical chemical sensor also includes a sensor element 902 disposed within a sensor well 302 of the well plate 210. The implantable optical chemical sensor also optionally includes a mask coating 904, and an optically transparent adhesive 906. In operation, light is emitted by the optical emitter 207 and into the well plate 210 and then into the sensor element 902. The light then passes through the sensor element 902 and before being redirected by a second opposing beveled facet 704 and onto optical detector 204.

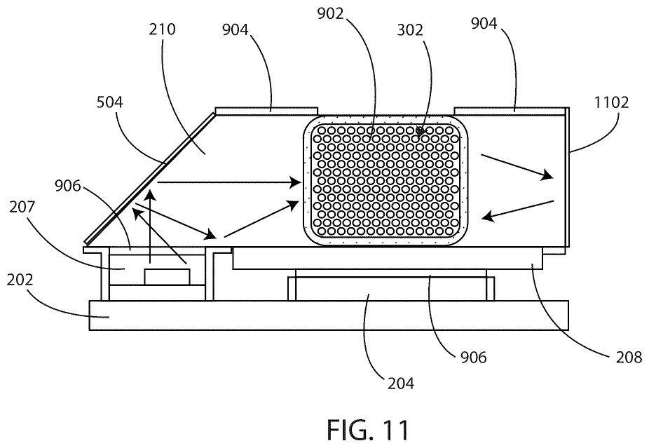

In some embodiments, light can reflect off a wall on one side of the well plate before returning to a sensor element and/or being received by an optical detector. For example, referring now to FIG. 11, a schematic cross-sectional view of components of an optical chemical sensor is shown in accordance with various embodiments herein. The implantable optical chemical sensor includes a circuit board 202, a first optical detector 204, and an optical emitter 207. The implantable optical chemical sensor also includes a floor plate 208 and a well plate 210. The well plate 210 includes a beveled facet 504. The implantable optical chemical sensor also includes a sensor element 902. The implantable optical chemical sensor also optionally includes a mask coating 904 and an optically transparent adhesive 906.

The implantable optical chemical sensor of FIG. 11 also includes a reflecting facet 1102, which may have a metallized or silvered coating thereon to promote reflection. In operation, light is emitted by the optical emitter 207 and into the well plate 210 and then into the sensor element 902. The light then passes through the sensor element 902 and onto the reflecting facet 1102, before it is reflected back to the sensor element 902. This reflection back can offer additional interaction between the light and the sensor element 902. Light can also scatter after interfacing with the sensor element 902 and travel downward to the optical detector 204.

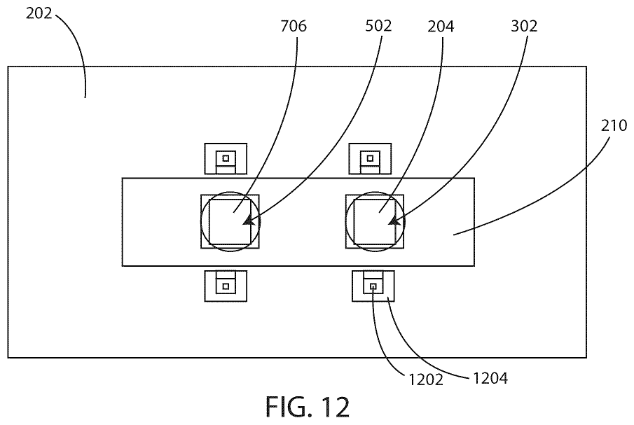

In some embodiments, one or more prisms can be used to redirect light (using refraction) from the emitters into the well plate and then into the sensor well and the sensor element disposed therein. Referring now to FIG. 12, a schematic plan view of an implantable optical chemical sensor 106 is shown in accordance with various embodiments herein. As before, the implantable optical chemical sensor can include a circuit board 202, a first optical detector 204, a second optical detector 706, a well plate 210 with a first sensor well 302 and a second sensor well 502.

The implantable optical chemical sensor 106 can also include prisms 1202 and prism holders 1204. The prisms 1202 can be aligned to receive light from the light emitters and guide the light into the well plate. For example, the prisms 1202 can be configured to change the direction of the light by approximately 90 degrees (such as with a right-angle prism), though other angles are also contemplated herein such as between 45 and 120 degrees. The prisms 1202 can have a silvered face so as to function like a mirror and aid in guiding the light into the well plate. The prisms 1202 can be formed of various materials including glasses, crystals, ceramics, polymers, and the like. The prisms can be of various sizes. In some embodiments, the prisms can be approximately 100, 200, 300, 400, 500, 600, 700, 800, 900, or 1000 microns on a side, or a size falling within a range between any of the foregoing. In some embodiments herein, mirrors or waveguides can be used in place of prisms 1202.

In some embodiments, the prism holders can be configured to both hold the prism and fit over the light emitter (such as an LED) providing a means for consistently aligning the prims with the light emitter. In some embodiments, the prism holders can be formed of a plastic material, though other materials are contemplated herein. The prism holders can hold the prisms utilizing a snap-fit mechanism and/or other means of fixation such as adhesive bonding and the like.

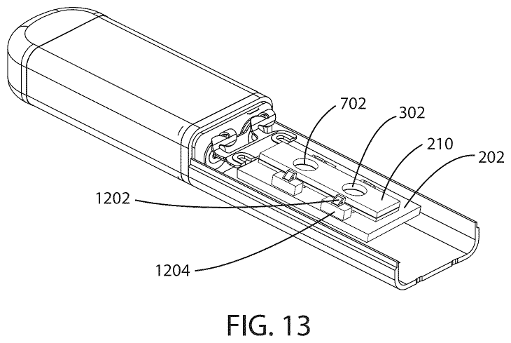

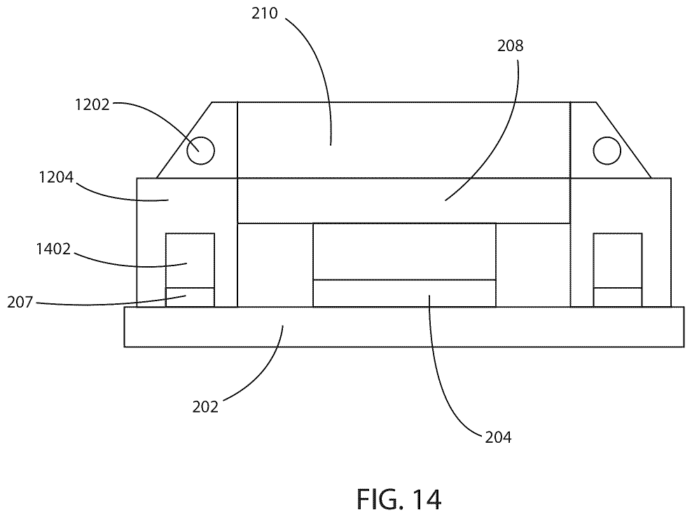

Referring now to FIG. 13, a schematic perspective view of portions of an implantable optical chemical sensor 106 is shown in accordance with various embodiments herein. As before, the implantable optical chemical sensor can include a circuit board 202, an optical emitter 207, a first optical detector 204, a second optical detector 706, and a well plate 210 with a first sensor well 302 and a second sensor well 502. The implantable optical chemical sensor also includes a prism 1202 and a prism holder 1204.

Referring now to FIG. 14, a schematic elevation view of portions of an implantable optical chemical sensor element is shown in accordance with various embodiments herein. As before, the implantable optical chemical sensor can include a circuit board 202, an optical emitter 207, a first optical detector 204, a second optical detector 706, a well plate 210 with a first sensor well 302 and a second sensor well 502. The implantable optical chemical sensor also includes a well plate 210 including a first sensor well 302 and a second sensor well 502. The implantable optical chemical sensor also includes a prism 1202 and a prism holder 1204. In this example, the implantable optical chemical sensor also optionally includes a waveguide 1402 which can be used to facilitate conveying light from the optical emitter to the respective prism.

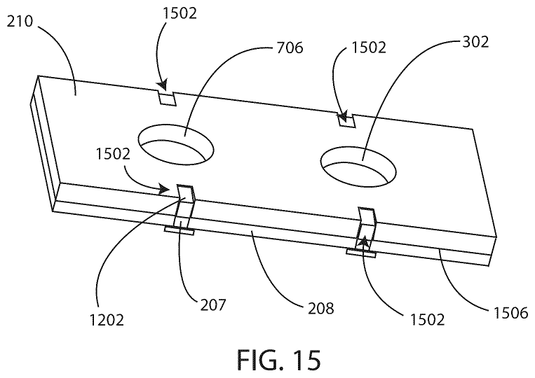

In some embodiments, the well plate and/or another component of the implantable optical chemical sensor (such as the floor plate) can include notches to aid in proper and consistent alignment of components. Referring now to FIG. 15, a perspective view of portions of an implantable chemical sensor element is shown in accordance with various embodiments herein. The implantable optical chemical sensor includes an optical emitter 207, a floor plate 208, a well plate 210 including a first sensor well 302. The implantable optical chemical sensor also includes a second optical detector 706. The implantable optical chemical sensor also includes a prism 1202.

In this embodiment, the well plate 210 also includes a prism alignment notch 1502. In various embodiments, the prism alignment notch 1502 can be configured to receive at least a portion of a prism 1202 or a structure connected thereto (such as a prism holder structure) thereby aligning the prism 1202 with a well plate 210. This can function to increase the consistency and precision of the interface between the prism 1202 and the well plate 210.

In some embodiments the implantable optical chemical sensor also includes a mask layer 1506 (or light blocking layer or occluding layer). The mask layer 1506 can be disposed underneath the well plate 210 and block the passage of light out of the bottom of the well plate 210. However, the mask layer 1506 can include apertures therein so as to allow light to pass out of the bottom of the sensor well and to an optical detector. The mask layer 1506 can include components such as carbon black or various pigments or dyes to be opaque to the passage of light. In some embodiments, the mask layer 1506 can be formed of a material such as black polyester.

### Chemical Sensing Chemistry

Various embodiments herein include a chemical sensing element which can include a chemical sensing composition. The chemical sensing composition can be part of a chemical sensing element herein. In some embodiments, the chemical sensing composition can be part of discrete physical elements such as beads within a chemical sensing element. Further details about the chemical sensing composition are provided as follows. However, it will be appreciated that this is merely provided by way of example and that further variations are contemplated herein.

In some embodiments, the chemical sensing composition of the chemical sensor element can be formed of a lipophilic indicator dye. In some embodiments, the chemical sensing composition can be formed from a lipophilic fluorescent indicator dye. In other embodiments, the chemical composition can be formed from a lipophilic colorimetric indicator dye. Suitable lipophilic indicator dyes can include, but are not limited to, ion selective sensors such as ionophores or fluorophores.

In some embodiments, ionophores herein can include, but not be limited to, sodium specific ionophores, potassium specific ionophores, calcium specific ionophores, magnesium specific ionophores, and lithium specific ionophores. In some embodiments, fluorophores can include, but not be limited to, lithium specific fluorophores, sodium specific fluorophores, and potassium specific fluorophores.

Chemical sensing compositions herein can include components (or response elements) that are configured for a response to an analyte of interest such as a colorimetric response, a photoluminescent response, or another optical sensing modality.

Colorimetric response elements herein can be specific for a particular chemical analyte of interest (such as potassium or another electrolyte, or various other compounds of medical/physiological interest). Colorimetric response elements can include an element that changes color based on binding with or otherwise complexing with a specific chemical analyte. In some embodiments, a colorimetric response element can include a complexing moiety and a colorimetric moiety. The colorimetric moiety can exhibit differential light absorbance on binding of the complexing moiety to an analyte.

Photoluminescent response elements herein can be specific for a particular chemical analyte. Photoluminescent response elements herein can include an element that absorbs and emits light through a photoluminescent process, wherein the intensity and/or wavelength of the emission is impacted based on binding with or otherwise complexing with a specific chemical analyte. In some embodiments, a photoluminescent response element can include a complexing moiety and a fluorescing moiety. Those moieties can be a part of a single chemical compound or they can be separated on two or more different chemical compounds. In some embodiments, the fluorescing moiety can exhibit different fluorescent intensity and/or emission wavelength based upon binding of the complexing moiety to an analyte.

Some chemistries may not require a separate compound to both complex an analyte of interest and produce an optical response. By way of example, in some embodiments, the response element can include an optical moiety or material wherein selective complexation with the analyte of interest directly produces either a colorimetric or fluorescent response. As an example, a fluoroionophore can be used and is a compound including both a fluorescent moiety and an ion complexing moiety. As merely one example, (6,7-[2.2.2]-cryptando-3-[2″-(5″-carboethoxy)thiophenyl]coumarin, a potassium ion selective fluoroionophore, can be used (and in some cases covalently attached to polymeric matrix or membrane) to produce a fluorescence-based K⁺ response element.

An exemplary class of fluoroionophores are the coumarocryptands. Coumarocryptands can include lithium specific fluoroionophores, sodium specific fluoroionophores, and potassium specific fluoroionophores. For example, lithium specific fluoroionophores can include (6,7-[2.1.1]-cryptando-3-[2″-(5″-carboethoxy) furyl]coumarin. Sodium specific fluoroionophores can include (6,7-[2.2.1]-cryptando-3-[2″-(5″-carboethoxy) furyl]coumarin. Potassium specific fluoroionophores can include (6,7-[2.2.2]-cryptando-3-[2″-(5″-carboethoxy) furyl]coumarin and (6,7-[2.2.2]-cryptando-3-[2″-(5″-carboethoxy)thiophenyl]coumarin.

Suitable fluoroionophores include the coumarocryptands taught in U.S. Pat. No. 5,958,782, the disclosure of which is herein incorporated by reference. Such fluorescent ionophoric compounds can be excited with GaN blue light emitting diodes (LEDs) emitting light at or about 400 nm. These fluorescent ionophoric compounds have ion concentration dependent emission that can be detected in the wavelength range of about 450 nm to about 470 nm.

These response elements can include complexing moieties. Suitable complexing moieties can include cryptands, crown ethers, bis-crown ethers, calixarenes, noncyclic amides, and hemisphere and moieties as well as ion selective antibiotics such as monensin, valinomycin and nigericin derivatives.

Those of skill in the art can recognize which cryptand and crown ether moieties are useful in complexing particular cations, although reference can be made to, for example, Lehn and Sauvage, “[2]-Cryptates: Stability and Selectivity of Alkali and Alkaline-Earth Macrocyclic Complexes,” J. Am. Chem. Soc, 97, 6700-07 (1975), for further information on this topic. Those skilled in the art can recognize which bis-crown ether, calixarene, noncyclic amides, hemispherand, and antibiotic moieties are useful in complexing particular cations.

By way of example cryptands can include a structure referred to as a cryptand cage. For cryptand cages, the size of the cage is defined by the oxygen and nitrogen atoms and the size makes cryptand cages quite selective for cations with a similar diameter. For example, a [2.2.2] cryptand cage is quite selective for cations such as K⁺, Pb⁺², Sr⁺², and Ba⁺². A [2.2.1] cryptand cage is quite selective for cations such as Nat and Ca⁺². Finally, a [2.1.1] cryptand cage is quite selective for cations such as Li⁺ and Mg⁺². The size selectivity of cryptand cages can aid in the sensitivity of chemical sensing. When these cryptand cages are incorporated into physiologic sensing systems heavier metals such as Pb⁺² and Ba⁺² are unlikely to be present in concentrations which interfere with the analysis of ions of broader physiological interest such as Na⁺ and K⁺.

### Optical Emitters and Detectors

In some embodiments, optical emitters herein can include solid state light sources such as GaAs, GaAlAs, GaAlAsP, GaAlP, GaAsp, Gap, GaN, InGaAlP, InGaN, ZnSe, or SiC light emitting diodes or laser diodes that can excite a chemical sensor element at or near the wavelength of maximum absorption for a time sufficient to emit a return signal. However, it will be understood that in some embodiments the wavelength of maximum absorption reflection varies as a function of concentration in the chemical sensor.

In some embodiments, the optical emitters can include other light emitting components including incandescent components. In some embodiments, the optical emitters can include a wave guide. The optical emitters can also include one or more bandpass filters, high pass filter, low pass filter, antireflection elements, and/or focusing optics.

In some embodiments, the optical excitation assembly can include a plurality of LEDs with bandpass filters, each of the LED-filter combinations emitting at a different center frequency. According to various embodiments, the LEDs can operate at different center-frequencies, sequentially turning on and off during a measurement, illuminating the chemical sensor element. As multiple different center-frequency measurements are made sequentially, a single unfiltered detector can be used in some embodiments. However, in some embodiments, a polychromatic source can be used with multiple detectors that are each bandpass filtered to a particular center frequency.

The chemical sensor element can include various types of ion selective sensor chemistries. Physiological analytes of interest can diffuse into and out of the chemical sensor element and bind with an ion selective sensor component to result in a fluorimetric or colorimetric response. Reference analytes can similarly diffuse into and out of the chemical sensor element and serve as a control sample. Exemplary ion selective sensors are described more fully below.

The optical detectors can be configured to receive light from the chemical sensor element. In an embodiment, the optical detectors can include a component to receive light. By way of example, in some embodiments, the optical detectors can include a charge-coupled device (CCD). In other embodiments, the optical detectors can include a photodiode, a junction field effect transistor (JFET) type optical sensor, or a complementary metal-oxide semiconductor (CMOS) type optical sensor. In some embodiments, the optical detectors can include an array of optical sensing components. In some embodiments, the optical detectors can include a waveguide.

The optical detectors can also include one or more bandpass filters and/or focusing optics. In some embodiments, the optical detectors can include one or more photodiode detectors, each with an optical bandpass filter tuned to a specific wavelength range. Signals from the optical detectors can be conveyed to a processor for analysis, such as a microprocessor which can perform various operations on the signals including detecting magnitudes of signal intensity, filtering operations, averaging signals, converting signals into concentrations of analytes of interest utilizing a predetermined correlation, or the like.

### Methods

Many different methods are contemplated herein, including, but not limited to, methods of making, methods of using, and the like. Aspects of system/device operation described elsewhere herein can be performed as operations of one or more methods in accordance with various embodiments herein.

For example, a method of using an implanted device to optically sense a chemical analyte can be included herein. The method can include operations of emitting light into a well plate, redirecting the light toward a sensor well-such as with a beveled facet, allowing the light to interface with a sensor element in the sensor well, and receiving light (in a scattering mode and/or in a transmission mode) with an optical detector and then processing a signal from the optical detector in order to determine the amount of a selected analyte.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An implantable optical chemical sensor comprising:
a circuit board;
at least one optical emitter, wherein the at least one optical emitter are disposed on the circuit board;
at least one optical detector, wherein the least one optical detector is disposed on the circuit board;
a floor plate; and
a well plate;
the well plate defining at least one well;
the well plate comprising
a vertical facet;
a beveled facet, wherein the beveled facet and the vertical facet are disposed on at least one perimeter edge of the well plate;
wherein the well plate is disposed over the floor plate;
a feed-through flange defining a window, wherein the vertical facet of the well plate fits into the window; and
wherein the at least one optical emitter is configured to emit light through the floor plate and into the well plate that is then redirected by the beveled facet of the well plate into the at least one well.

2. The implantable optical chemical sensor of claim 1, wherein the beveled facet is disposed at an angle of 20 to 70 degrees with respect to a surface of the vertical facet.

3. The implantable optical chemical sensor of claim 1, further comprising a sensor element, wherein the sensor element is configured to fit into the at least one well.

4. The implantable optical chemical sensor of claim 1, further comprising a coating, wherein the coating is disposed over the beveled facet and configured to cause internal reflection of light off the beveled facet.

5. The implantable optical chemical sensor of claim 1, wherein the floor plate includes a low-index glass.

6. The implantable optical chemical sensor of claim 1, wherein the at least one optical detector comprises a photo-diode.

7. The implantable optical chemical sensor of claim 1, the implantable optical chemical sensor further comprising a top housing shell, the top housing shell comprising a sensor window, wherein the feed-through flange fits into the sensor window.

8. The implantable optical chemical sensor of claim 7, wherein a top surface of the feed-through flange fits flush with a top surface of the top housing shell.

9. An implantable optical chemical sensor comprising:
a circuit board;
at least one optical emitter, wherein the at least one optical emitter is disposed on the circuit board;
a floor plate; and
a well plate, the well plate defining at least one well;
wherein the well plate is disposed over the floor plate;
at least one prism, wherein the at least one prism is arranged to receive light from the at least one optical emitter and direct the same into the well plate;
at least one optical detector, wherein the least one optical detector is disposed on the circuit board; and
a feed-through flange, the feed-through flange defining a window.

10. The implantable optical chemical sensor of claim 9, further comprising a prism holder, wherein the prism holder is configured to hold a prism and fit over at least one optical emitter thereby aligning the same.

11. The implantable optical chemical sensor of claim 9, further comprising a sensor element, wherein the sensor element is configured to fit into the at least one well.

12. The implantable optical chemical sensor of claim 9, wherein the floor plate includes a low-index glass.

13. The implantable optical chemical sensor of claim 9, further comprising:
a top housing shell, the top housing shell defining a sensor window; and
wherein the feed-through flange fits into the sensor window.

14. The implantable optical chemical sensor of claim 13, wherein a top surface of the feed-through flange fits flush with a top surface of the top housing shell.

15. An implantable optical chemical sensor comprising:
a circuit board;
at least one optical emitter, wherein the at least one optical emitter is disposed on the circuit board;
at least one optical detector, wherein the least one optical detector is disposed on the circuit board;
at least one prism; and
a well plate, the well plate comprising
at least one well; and
at least one prism alignment notch, wherein the at least one prism alignment notch is configured to receive at least a portion of the at least one prism or a structure connected thereto thereby aligning the prism with the well plate;
a feed-through flange, the feed-through flange defining a window; and
wherein the at least one prism is arranged to receive light from the at least one optical emitter and direct the same into the well plate.

16. The implantable optical chemical sensor of claim 15, further comprising a prism holder, wherein the prism holder is configured to hold a prism and fit over at least one optical emitter thereby aligning the same.

17. The implantable optical chemical sensor of claim 15, further comprising a sensor element, wherein the sensor element is configured to fit into the at least one well.

18. The implantable optical chemical sensor of claim 15, further comprising a floor plate, wherein the well plate is disposed over the floor plate.

19. The implantable optical chemical sensor of claim 15, further comprising:
a top housing shell, the top housing shell defining a sensor window; and
wherein the feed-through flange fits into the sensor window.

20. The implantable optical chemical sensor of claim 19, wherein a top surface of the feed-through flange fits flush with a top surface of the top housing shell.
